(19) United States
(12) Patent Application Publication
(10) Pub. No.: US 2016/0135974 A1
(43) Pub. Date: May 19, 2016

(54) REDUCED FRICTION INTRODUCER ASSEMBLY

(71) Applicant: COOK MEDICAL TECHNOLOGIES LLC, BLOOMINGTON, IN (US)

(72) Inventor: Anders Ginge Jensen, Hornslet (DK)

(73) Assignee: Cook Medical Technologies LLC

(21) Appl. No.: 14/945,566

(22) Filed: Nov. 19, 2015

(30) Foreign Application Priority Data
Nov. 19, 2014 (GB) 1420561.1

(51) Int. Cl. A61F 2/966

(52) U.S. Cl. CPC A61F 2/966

(57) ABSTRACT

A medical implant delivery device includes an elongate carrier which includes a device holding member at its distal end, attached to a catheter element. The device holding member includes at least one channel extending along its length. A pusher element, in the form of an outer catheter, is disposed over the elongate carrier and provides a pusher shoulder. A medical device can be held on the device holding member. An outer sheath is typically disposed over the implant delivery device, in known manner. The device holding member is made of a resilient material able to compress radially inwardly, particularly by compression of the walls bounding the channel. The compressibility of the device holding member reduces the friction between the medical device and the carrier sheath as well as the force required to retract the outer sheath during the deployment of the medical device.

### CROSS-REFERENCE TO RELATED APPLICATIONS

This application claims the benefit of priority under 35 U.S.C. §119(a) to Great Britain Patent Application No. GB 1420561.1, filed Nov. 19, 2014, which is hereby incorporated by reference in its entirety.

### TECHNICAL FIELD

The present invention relates to an introducer assembly and in the preferred embodiment to a medical implant delivery device.

### BACKGROUND ART

Introducer assemblies are in common use for carrying out endovascular medical procedures, such as implantation of a temporary or permanent medical implant. These assemblies typically include an outer sheath or catheter which holds and protects the components of the assembly, including the medical device, while they are pushed endoluminally through the patient's vasculature to the remote treatment site. The assembly typically also includes a carrier element, which is often also a catheter, on which the medical device is held and carried during the introduction procedure. The carrier element is typically provided with one or more device holding elements which can be actuated to release the medical device into a vessel of the patient.

Such introducer assemblies are often required to pass through tortuous paths, which requires them to be flexible and yet sufficiently strong in the longitudinal direction to provide pushability from a remote percutaneous entry point. The outer sheath or catheter is advantageously as small in diameter as possible, in order to improve endovascular movement and also in order to hold the medical device properly in position until its deployment. As a result, the medical device is normally tightly held in the outer sheath or catheter, this being especially so in the case of self-expanding medical devices.

There is typically experienced a notable friction at the start of the release procedure, when the outer sheath is first retracted to expose the medical device at the distal end of the carrier element. This friction can lead to difficulties in deploying the medical device, risks in misplacement of the device and also in damage or twisting of the device as the sheath is pulled back.

Examples of prior art introducer assemblies can be found in U.S. Pat. No. 3,826,256, 4,601,713, US-2005/027,282, US-2009/204,082, US-2010/024,830 and WO-02/49,713.

### DISCLOSURE OF THE INVENTION

The present invention seeks to provide an improved introducer assembly. The preferred embodiments are directed to an introducer assembly for deploying an implantable medical device. However, the teachings herein can be used also for other types of introducer assemblies, for instance assemblies used to carry medical or diagnostic tools.

According to an aspect of the present invention, there is provided a medical implant delivery device including: an elongate carrier including a distal section and a proximal section, the distal section including a carrier zone for carrying an implantable medical device thereon; the distal section of the carrier extending in a longitudinal direction and having an elongate recess extending in the longitudinal direction, the distal section of the carrier being made of a resilient material enabling compression of the carrier in a direction transverse to the longitudinal direction.

The structure enables the distal section of the carrier to compress radially inwardly in the transverse direction, in effect increasing the space within a carrier sheath for an implantable medical device disposed on the elongate carrier. Increasing this space has the effect of reducing the force of the medical device against the internal wall of the outer sheath, thereby reducing the friction between the outer sheath and the medical device. This reduction in friction facilitates the deployment of the medical device, reduces the chance of incorrect placement of the device and reduces the chance of damage to the device as the sheath is slid over the device during its retraction.

Advantageously, the elongate recess provides a channel in the distal section. The elongate recess may be U-shaped.

In the preferred embodiment, the distal section has a wall shaped to provide the recess, the wall having a generally uniform thickness. The wall of the distal section may be of solid material.

Advantageously, the distal section includes a lumen or channel for the passage of a guide wire or catheter therethrough.

In a preferred embodiment, the distal section is coated with a low friction material. This may be polytetrafluoroethylene, or a hydrophilic material.

In a practical embodiment, the device may include a strengthening wire embedded within the distal section, the wire preferably being of metal. The wire may be located in a side wall portion of the distal section.

In a preferred embodiment, the proximal section includes a catheter which is substantially round in transverse cross-section, a distal end of the catheter being attached to a proximal end of the distal section, the catheter having a lumen therein for the passage of a guide wire.

The device may include a pusher element of elongate form having a lumen therein, wherein the elongate carrier is disposable in the lumen of the pusher element.

The assembly preferably includes an outer sheath disposable over the carrier and pusher element. In other examples, the assembly could be introduced into an outer sheath, for instance one already implanted into a patient.

Other features and advantages of the teachings herein will become apparent from the specific description which follows.

### BRIEF DESCRIPTION OF THE DRAWINGS

Embodiments of the present invention are described below, by way of example only, with reference to the accompanying drawings, in which:

FIG. 1 is a schematic perspective view of the distal end of an embodiment of medical implant delivery device; and

FIG. 2 is a schematic perspective view of an embodiment of introducer assembly including a delivery device as shown in FIG. 1.

### DESCRIPTION OF THE PREFERRED EMBODIMENTS

It is to be understood that the drawings are schematic only and not to scale. They are intended to depict the major components of the devices taught herein and minor or ancillary components are not shown for the sake of clarity. The skilled person will be well acquainted with the typical dimensions and proportions suitable for the components and devices shown in the drawings, particularly having regard to the accompanying description.

The preferred embodiments described below are directed to a medical implant delivery device and the examples show a stent or stent graft. It is to be understood that the teachings herein are not limited to a particular medical device or type of medical device and that the device carrier components could be readily modified to carry other types of medical device. Furthermore, the teachings herein can also be adapted for medical tooling and diagnostic tooling rather than for the deployment of implantable medical devices.

Only the principal components of the assembly taught herein are described below and shown in the drawings, for the sake of clarity. In particular, the drawings do not show device constraining elements typically used to hold the medical device radially compressed on the holding element, such as trigger wires, holding sleeves and the like. These elements are conventional in the art and will therefore be readily envisioned by the skilled person.

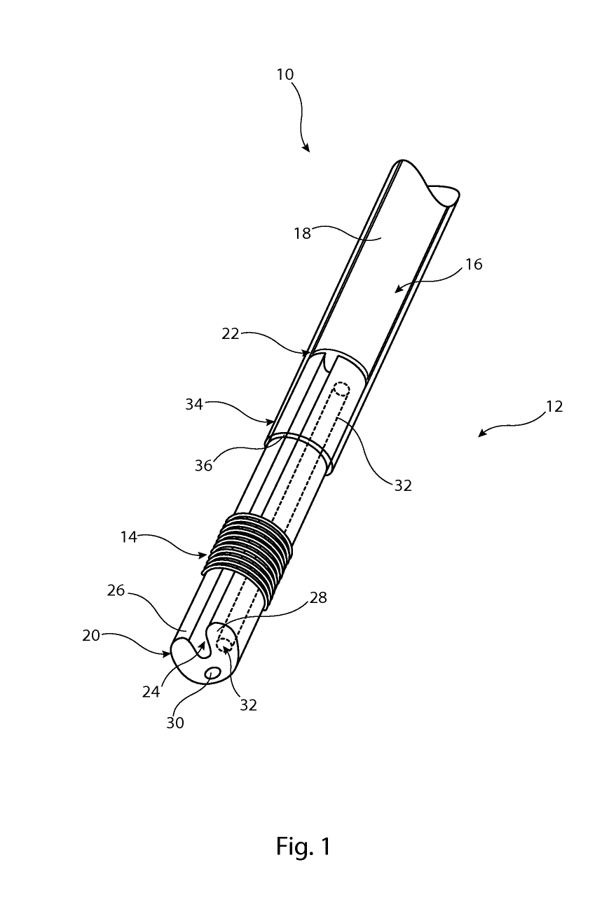

Referring first to FIG. 1, this shows the distal end 12 of the preferred embodiment of medical implant delivery device 10.

In the view of FIG. 1, the device 10 is shown carrying a medical implant 14, which may be a stent, stent graft or any other medical device which is substantially tubular in shape when carried on the delivery device 10. The skilled person will appreciate that the device 10 could be modified to carry other shapes of medical device.

The delivery device 10 includes an elongate carrier 16, which in this embodiment is formed of two components, the first being a catheter element 18 and the second being a device holding member 20 which is attached at the distal end 22 of the catheter element 18 so as to be fixed to or integral with the catheter element 18. In the preferred embodiment, the catheter element 18 includes at least one lumen extending therethrough (not visible in FIG. 1) for the passage of a guidewire (not shown). It is not excluded, though, that in the other embodiments the catheter element may have no lumens therein and thus be in the form of a flexible rod.

The catheter element 18 may be made of any suitable material and specifically of a commonly used material for catheters, and may also be provided with a strengthening element therein for providing the catheter 18 with longitudinal strength for pushability as well as bending strength to prevent kinking of the device 10 when fed through tortuous vasculature. As suitable structures for such a catheter element 18 will be readily familiar to the person skilled in the art they are not described in any further detail herein.

The device holding member 20 is, in this embodiment, U-shaped in transverse cross-section, so as to have a channel 24 therewithin, which in this embodiment runs through the length of the holding member 20, terminating at the distal end 22 of the catheter element 18. The channel 24 is bounded by first and second facing side walls 26, 28 of the device holding member 20, which are thereby spaced from one another by the opening provided by the channel 24. The device holding member 20 is preferably made of solid material, having in this embodiment having a lumen 30 extending therethrough, which is aligned with a lumen (not visible) within the catheter element 18 for the passage of a guide wire through the device 10.

Embedded within the holding member 20 is a stabilisation wire 32, which is fixed to the material of the holding member 20, for example by bonding or on molding or casting of the member 20. In the embodiment shown, the stabilisation wire 32 is disposed within the thickness of the sidewall 28. The stabilisation wire 32 could be disposed elsewhere within the volume of the holding member 20 and in some embodiments there may be provided more than one stabilisation wire. It is preferred, as shown in FIG. 1, that the stabilisation wire 32 extends through the whole length of the device holding member 20 and may in some embodiments extend into at least a part of the catheter element 18 and be embedded or attached to the catheter element 18, in such a manner as to make use of the strength of the catheter element 18 in supporting the stabilisation wire 22 and hence the device holding member 20. The stabilisation wire 32 may be made of any suitable material, typically a metal or metal alloy. It could also be made of a polymer material which is preferably substantially inextensible and which has high compressive strength in the longitudinal direction.

The purpose of the stabilisation wire 32 is to give longitudinal integrity to the device holding member 20, that is to resist any longitudinal extension or compression of the device holding member 20, particularly during the deployment process of the device 10.

The device holding member 20 is made of a polymeric amide such as nylon, polyurethane or other suitable material. The use of a resilient material and provision of a channel 24 enables the holding member 20 to compress in the transverse direction, preferably by pressing the side walls 26 and 28 towards one another.

The delivery device 10 also includes, in this embodiment, an outer catheter 34, which is disposed over the catheter element 18 and a section of the holding member 20 as will be apparent in FIG. 1. The outer catheter 24 includes a distal end 36 which provides a flat shoulder in the transverse direction, acting as a pusher element against which the medical device 14 can be held in the longitudinal direction. The distal end 36 stops the medical device 14 from sliding down the delivery device 10 during the deployment process.

The outer catheter 34 may be fixed in position relative to the catheter element 18 and device holding member 20, although in other embodiments it may be slidable relative to these two elements, so as to be able to push the medical device 14 in a distal direction off the device holding member 20 during the deployment process. Typically, though, the outer catheter 34 is fixed in position.

The outer catheter 34 may be omitted in some embodiments, with the pusher shoulder being provided by having a catheter element 18 with a diameter greater than the diameter of the device holding member 20 so as to extend laterally beyond the periphery of the holding member 20, thereby providing the desired stop shoulder.

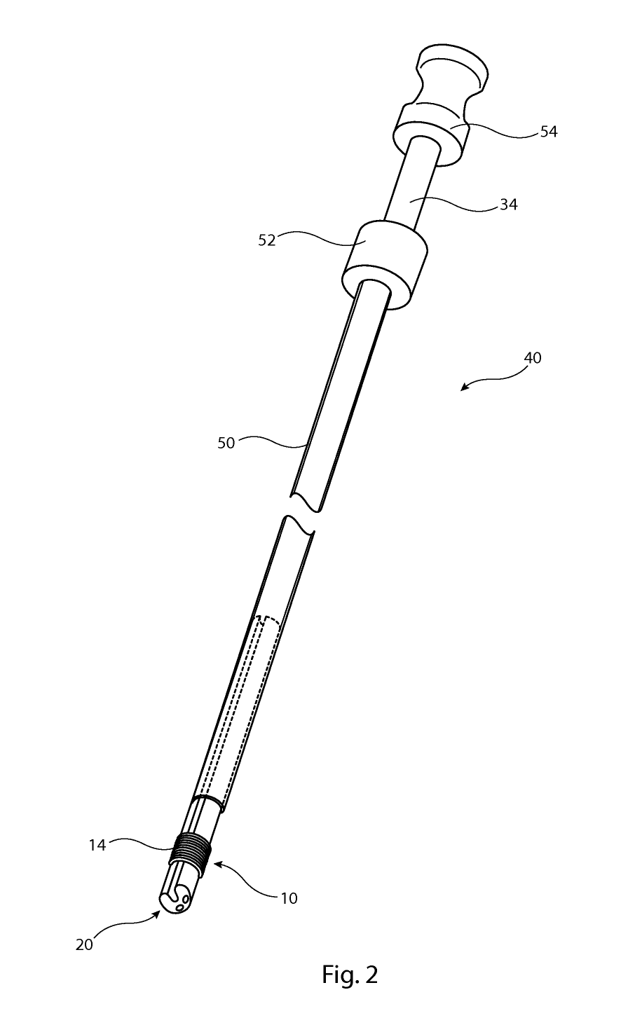

Referring now to FIG. 2, this shows an example of introducer assembly 40 which includes the implant delivery device 10 shown in FIG. 1. The introducer assembly 40 also includes an outer sheath 50 which is disposed concentrically over the implant delivery device 10 and specifically over the outer catheter 34. When in the distal-most position, the outer sheath 50 covers also the device holding member 20 and the medical device 14 carried thereon. At the proximal end of the sheath 50 there is provided a handle element 52, while the implant delivery device includes own handle element 54. Suitable handle mechanisms are well known in the art. The outer sheath 50 is slidable over the implant delivery device 10, typically by holding the two handle portions 52, 54 and applying the appropriate pressure.

Not shown in FIG. 2 but typically provided with the assembly 40 is a dilator element, attached to the distal end of the device holding member 20, which can be of known type. The dilator element may have a lumen passing therethrough, which can be aligned with the lumen 30 of the device holding member 20, for the passage of a guidewire through the introducer assembly 40, for purposes known in the art.

The outer sheath 50 is typically disposed during the deployment procedure so as to cover the distal end of the device 10 and in particular the medical device 14, thereby protecting the medical device 14 during the endoluminal insertion and feeding process.

As with such introducer assemblies, it is preferred that the assembly 40 has the smallest footprint possible, that is the smallest transverse diameter, which improves the trackability of the assembly through the patient's vessels. Furthermore, it is advantageous that the components of the assembly 40 are a tight fit, which assists in holding the medical device 14 precisely to the device holding member 20 and in preventing any variation in the shape or configuration of the medical device 14 during the endoluminal passage of the assembly 40 to the treatment site.

As a result of the tight fit, the medical device 14 will normally be pressed against the internal wall of the sheath 50, as well as against the outer surface of the device holding member 20. In a conventional introducer apparatus, when the device holding member is a catheter of round or substantially round or cross-section, the catheter is not able to compress radially to any great extent, leading to increased pressure, and therefore increased friction, of the medical device against the internal surfaces of the sheath 50. This makes it necessary to apply a greater sheath withdrawal force in order to pull the sheath 50 back so as to expose the medical device, which also increases the risk of deformation or damage to the medical device 14. By contrast, the structure of the device holding member 20, and in particular its ability to compress radially inwardly, contributes to a reduction in the force imparted on the medical device 14 and consequently a reduction in the friction between the medical device 14 and the sheath 15. As a result, less force is necessary to retract the sheath 50 so as to expose a medical device 14. Furthermore, the reduction in friction also reduces the forces applied to the medical device 14 during the sheath retraction process.

The skilled person will appreciate that the radial compressibility of the device holding element 20 enables the medical device to extend radially inwardly as the result of pressures applied thereto, both by the sheath 50 per se but also in circumstances in which the medical device 14 is deployed in a curved lumen, when it can be expected that the distal end of the assembly 40 including the device holding member may be caused to be curved.

In the preferred embodiment, the device holding member 20 is coated to reduce friction with the medical device 14. The coating could be of polytetrafluoroethylene (PTFE), or a suitable hydrophilic coating.

The preferred embodiment has a channel 24 which is U-shaped, although the person skilled in the art will appreciate that the channel could have other shapes including internal walls which are substantially flat, converging to an apex, in other words V-shaped. Similarly, in other embodiments, there may be provided a plurality of channels extending along the length of the device holding member, typically 2, 3, 4 or more such channels.

In the embodiment shown, the device holding member 20 forms only the distal end of the elongate carrier 16, with the majority of the carrier 16 being formed of the catheter element 18. In other embodiments, the entirety of the elongate carrier 16 could be formed as a channelled element, in some instances being singularly the device holding member 20 and therefore compressible along its entire length, while other embodiments may be formed of different materials so as to be less compliant along the majority of its length proximally of the distal holding section of the medical device. This can be achieved by use of different materials or by providing hardening additives in the more proximal section. The embodiments described above are used in the deployment of an implantable medical device such as a stent or stent graft. The skilled person will appreciate, though, that the structures disclosed herein could be used and if necessary adapted to deploy other types and shapes of medical device.

It is envisaged that the teachings herein, in particular the provisions of a channel holding member or structure of the type disclosed herein, could also be used for other purposes. In one example, the structure could be used as a channelled catheter holding a medical balloon, in which part of the uninflated balloon wall can be stored within the channel, which can assist in holding the balloon in position as well as during its deployment. Similarly, the channel could be used to hold parts of implantable medical devices, including barbs and so on.

All optional and preferred features and modifications of the described embodiments and dependent claims are usable in all aspects of the invention taught herein. Furthermore, the individual features of the dependent claims, as well as all optional and preferred features and modifications of the described embodiments are combinable and interchangeable with one another.

The disclosure in the abstract accompanying this application is incorporated herein by reference.

1. A medical implant delivery device including:
an elongate carrier including a distal section and a proximal section, the distal section including a carrier zone for carrying an implantable medical device thereon; the distal section of the carrier extending in a longitudinal direction and having an elongate recess extending in the longitudinal direction, the distal section of the carrier being made of a resilient material enabling compression of the carrier in a direction transverse to the longitudinal direction.

2. A device according to claim 1, wherein the elongate recess provides a channel in the distal section.

3. A device according to claim 1, wherein the elongate recess is U-shaped.

4. A device according to claim 1, wherein the distal section has a wall shaped to provide the recess, the wall having a generally uniform thickness.

5. A device according to claim 1, wherein the distal section includes a lumen or channel for the passage of a guide wire or catheter therethrough.

6. A device according to claim 1, wherein the distal section is coated with a low friction material.

7. A device according to claim 6, wherein the distal section is coated with polytetrafluoroethylene.

8. A device according to claim 6, wherein the distal section is coated with a hydrophilic material.

9. A device according to claim 1, including a strengthening wire embedded within the distal section.

10. A device according to claim 9, wherein the strengthening wire is of metal.

11. A device according to claim 9, wherein the wire is located in a side wall portion of the distal section.

12. A device according to claim 1, wherein the distal end is of solid material.

13. A device according to claim 1, wherein the proximal section includes a catheter which is substantially round in transverse cross section, a distal end of the catheter being attached to a proximal end of the distal section, the catheter having a lumen therein for the passage of a guide wire.

14. A device according to claim 1, including a pusher element of elongate form having a lumen therein, wherein the elongate carrier is disposable in the lumen of the pusher element.

15. A device according to claim 14, including an outer sheath disposable over the carrier and pusher element.
